(19) United States
(12) Patent Application Publication
(10) Pub. No.: US 2025/0049775 A1
(43) Pub. Date: Feb. 13, 2025

(54) USE OF AMINOPYRAZOLE COMPOUNDS

(71) Applicant: Intervet Inc., Rahway, NJ (US)

(72) Inventors: Linda Horspool, Ledeacker (NL); Timothy Kowalski, Plainsboro, NJ (US); Christina Moeller, Bischofsheim (DE)

(73) Assignee: Intervet Inc., Rahway, NJ (US)

(21) Appl. No.: 18/721,916

(22) PCT Filed: Dec. 23, 2022
(86) PCT No.: PCT/EP2022/087698

§ 371 Date: Jun. 20, 2024

(30) Foreign Application Priority Data
Dec. 24, 2021 (EP) 21217736.4

(51) Int. Cl. A61K 31/4439; A61K 45/06; A61P 17/04

(52) U.S. Cl. CPC A61K 31/4439; A61K 45/06; A61P 17/04

(57) ABSTRACT

The current invention provides the use compounds of Formula (I) which are JAK inhibitors, and as such for the treatment of JAK-1 mediated diseases such as atopic—and allergic dermatitis in young animals.

### TECHNICAL FIELD

This invention relates to a compound for use in the treatment of JAK-1 mediated diseases of an animal such as e.g., atopic dermatitis or allergic dermatitis.

### BACKGROUND OF THE INVENTION

Allergic diseases are one of the most serious problems of companion animals such as dogs, cats and horses. Symptoms of such diseases impacts their health and quality of life.

The potential factors involved in allergic diseases are numerous and poorly understood. These diseases are related to an allergic reaction that is a disorder or disease caused by an interaction between the immune system and a substance foreign to the body. This foreign substance is termed “an allergen”. Common allergens include aeroallergens, such as pollens, dust, molds, dust mite proteins, injected saliva from insect bites, etc.

The mammalian Janus kinase (JAK) family of non-receptor tyrosine kinases has four members; JAK-1, JAK-2, JAK-3 and TYK-2. The JAK family is involved in intracellular signal transduction from >70 different cytokines. Cytokines bind to their cell surface receptors resulting in receptor dimerization and subsequent activation/phosphorylation of JAK tyrosine kinases. Specific tyrosine residues on the receptor are then phosphorylated by activated JAKs and serve as docking sites for STAT proteins. STATs are phosphorylated by JAKs, dimerize, then translocate to the nucleus where they bind specific DNA elements and activate gene transcription. JAK-1 signals in conjunction with all JAK isoforms in a cytokine dependent manner.

JAKs are essential for multiple physiological functions. This is evidenced by studies using genetically engineered mouse models that are deficient in specific JAKs (K. Ghoreschi, A. Laurence, J. J. O'Shea, Immunol. Rev. 228,273 (2009)), and the identification of mutations in the JAK enzymes that have been associated with diseases in humans. (J. J. O'Shea, M. Pesu, D. C. Borie, P. S. Changelian, Nat. Rev. Drug Discov. 3, 555 (2004)). (Y. Minegishi et al., Immunity. 25, 745 (2006)).

These mouse and human genetic data link the Jak/STAT pathway to various diseases and disorders including but not limited to hyperproliferative disorders and cancer such as leukemia and lymphomas, immunological and inflammatory disorders such as transplant rejection, asthma, chronic obstructive pulmonary disease, allergies, rheumatoid arthritis, allergic and atopic dermatitis, type I diabetes, amyotropic lateral sclerosis and multiple sclerosis.

Recently the role of the mammalian Janus kinases (JAKs) in the pathophysiology of allergy has been investigated and better understood. A commercially available JAK inhibitor Apoquel®, an animal drug whose active ingredient is oclacitinib maleate, has been authorized for the control of pruritus associated with atopic dermatitis and control of atopic dermatitis in dogs. Oclacitinib is a partially selective JAK-1 inhibitor.

JAK inhibitors can inhibit the function of a variety of cytokines dependent on JAK enzyme activity. A number of these cytokines have a role in the pathophysiology of allergic skin disease/canine atopic dermatitis. While some of these cytokines are proinflammatory or implicated in allergic responses/pruritus, these and others exert a wide range of responses in a variety of cell types. For example, a number of JAK-dependent cytokines are important components of host defense or have a role in normal hematopoiesis.

Therefore, while JAK inhibitors may have utility in diseases such as atopic dermatitis that involve the dysregulation of cytokine signaling, there is potential for a range of other non-target effects (e.g., unwanted effects on host defenses, hematopoiesis, etc.).

Important known veterinary JAK-1 mediated diseases include conditions or diseases such as e.g., allergic diseases or conditions of the skin, gastrointestinal and respiratory tract. Examples are allergic reactions, allergic dermatitis, atopic dermatitis, eczema, summer eczema, urticaria, heaves, inflammatory airway disease, recurrent airway obstruction, airway hyper-responsiveness, chronic obstructive pulmonary disease, inflammatory processes resulting from autoimmunity and inflammatory diseases of non-human animals, especially of companion animals.

A number of JAK-1 mediated diseases include a pruritic condition, a disease or disorder characterized by an intense itching sensation that produces the urge to rub or scratch the skin to obtain relief. Animals generally display the sensation of itch clinically by scratching. However, it may also be manifested by licking, sucking, biting, chewing, rubbing, or rolling.

When the pruritus remains uncontrolled, especially over a longer period often secondary skin conditions complicate the treatment.

Skin conditions resulting from pruritus in animals can be e.g., alopecia, scaling and hyperpigmentation, erythema, hyperpigmentation, scaling, crusting, papules, and pustules. In younger dogs (or old dogs with a longstanding history dating from an onset of the disease as a young animal), it may present as chronic or chronic relapsing pyoderma.

The prevalence of atopic dermatitis is estimated to be 10% of the total canine population. Globally, about 4.5 million dogs are affected with this chronic and lifelong condition, and incidence appears to be increasing. The most frequently observed clinical sign is intense pruritus, which significantly affects the quality of life of both animals and their owners. The diagnosis is clinical, based on the history and characteristic clinical signs associated with the exclusion of other pruritic dermatopathies. Canine breed and sex predilections have been suspected but may vary greatly depending on geographical region.

Allergic dermatitis presents with multiple cutaneous reaction patterns that all may be caused by environmental, food and/or insect allergens, as well as other diseases. Allergic dermatitis is thought to be caused by an abnormal response of the immune system to substances that do not induce a reaction in healthy cats or dogs. The most consistent feature of allergic dermatitis is chronic recurrent pruritus. The potential factors involved in allergic dermatitis are numerous and poorly understood.

Current treatments depend on the severity of the clinical signs, duration, and owner preferences and include allergen-specific immunotherapy and antipruritic drugs such as glucocorticoids and cyclosporines. Immunosuppressive drugs like glucocorticoids and cyclosporines are generally effective however long-term use often results in undesirable adverse effects. While these drugs are effective, they have significant side effects that can prevent their long-term use. For example, these drugs suppress the animal's immune system, which can lead to infections. Corticosteroids also can cause osteoporosis, endocrine problems and cataracts in canines and felines, as well as other non-human animals. In addition, corticosteroids tend to cause animals to eat, drink and urinate frequently, which is considered undesirable by pet owners

Immunotherapy treatment is effective for some patients but requires frequent injections, and clinical improvement may not be seen for 6-9 months.

A recently approved product known as a Janus kinase, or JAK, inhibitor is also available for treating atopic dermatitis.

The JAK-1 enzyme is involved in signaling and signal transduction of pro-inflammatory, pro-allergic and pruritogenic cytokines associated with atopic dermatitis.

In dogs with allergic or atopic dermatitis, the administration of oclacitinib, produces a rapid amelioration of pruritus and reduces lesions [Cosgrove et al., Vet. Derm. (2013), 24:479; Cosgrove et al., Vet. Derm. (2013), 24:587]. At higher doses, oclacitinib produces a reduction in hematocrit, hemoglobin, and reticulocyte counts, presumably due to inhibition of JAK-2 [FOI Summary NADA 141-345; Gonzales et al., J. Vet. Pharmacol. Therapeut. (2014), 37:317].

Collectively, these data strongly suggest that inhibition of JAK-1 is an effective treatment for allergic and atopic dermatitis.

As atopic dermatitis in canines and felines is often a chronic condition, these safety and side effect issues create a significant unmet medical need for a safe and effective long-term treatment.

Alternative JAK Inhibitors are known. WO 2013/041042 discloses pyrazole carboxamdines as Janus Kinase Inhibitors that are useful for the treatment of rheumatoid arthritis, asthma, COPD and cancer. The compounds of this disclosure are of the following Formula

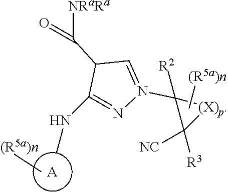

WO2013/040863 discloses substituted cycloalkylnitrile pyrazole carboxamides that are Janus kinase-1 inhibitors useful for treating e.g., asthma, obstructive airways diseases, arthritis, emphysema, cancer, myasthenia gravis, Graves' disease, and Alzheimer's disease.

WO2014/146490 discloses substituted 2-(3-amino-4-oxo-4,5-dihydro-pyrazolo(4,3-c) pyridin-1-yl)-cyclobutanecarbonitrile compounds are Janus kinase inhibitors.

WO 2018/108969 discloses compounds which are selective Janus-1 kinase (JAK) inhibitors, and as such are useful for the treatment of JAK-mediated diseases such as atopic dermatitis, arthritis, and cancer. Specifically, 1-[(3R,4S)-4-cyanotetrahydropyran-3-yl]-3-[(2-fluoro-6-methoxy-4-pyridyl) amino]pyrazole-4-carboxamide as compound of Formula (I) is disclosed.

Apoquel® is an animal drug whose active ingredient is oclacitinib which is authorized for the control of pruritus associated with atopic dermatitis and control of atopic dermatitis in dogs at least 12 months of age (See FOI Summary for NADA 141-345, May 14, 2013). See also U.S. Pat. Nos. 6,890,929; 7,687,507; 8,133,899 and 8,987,283.

For Apoquel a margin of safety (MOS) study was conducted. This GLP study was conducted in young adult beagle dogs, approximately 6 months old at study initiation. Oclacitinib was administered to dogs at 0, 1, 3, and 5 times the maximum exposure dose of 0.6 mg/kg twice per day. The intended duration of the study was 26 weeks. After 4 months of treatment, two dogs required euthanasia and several dogs in the mid and high dose groups developed clinical demodicosis which was attributed to treatment-related immunosuppression. Infection secondary to immunosuppression resulted in two deaths, including one death that occurred 28 days after discontinuation of oclacitinib maleate.

The study was prematurely terminated at 16 weeks.

It was found that the most severe adverse clinical effects were observed in dogs aged less than one year. As a result of the effects seen in young dogs, the decision was made to limit its use to dogs of greater than 1 year of age.

Therefore, the only available JAK-1 inhibitor Apoquel is contraindicated for use in animals below 12 months of age.

Considering the currently unmet need for safe and effective alternative treatments for atopic and allergic dermatitis in companion animals, especially for animals that are younger than 12 months of age it would be desirable to provide a new effective and safe treatment option.

### SUMMARY OF THE INVENTION

The current invention is directed to a compound of Formula (I) or a pharmaceutically acceptable salt or stereoisomer thereof

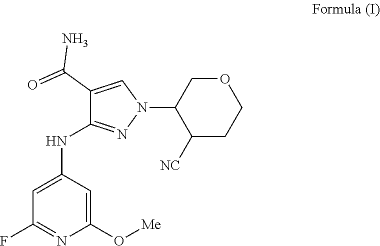 [SMILES: COc1cc(Nc2nn(C3COCCC3C#N)cc2C(N)=O)cc(F)n1]

for use in the treatment of an JAK-1 mediated disease of a companion animal selected from dog, cat, and horse, characterized in that a therapeutically effective dose of such compound is administered to the animal that is younger than 12 months of age.

Another embodiment is a pharmaceutical composition comprising a therapeutically effective dose of a compound of Formula (I)

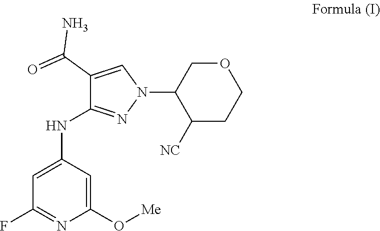 [SMILES: COc1cc(Nc2nn(C3COCCC3C#N)cc2C(N)=O)cc(F)n1]

and a pharmaceutically acceptable carrier for the use as indicated above.

Another aspect is a pharmaceutical composition comprising therapeutically effective dose of a compound of Formula (I) or a pharmaceutically acceptable salt or stereoisomer thereof as described above and a pharmaceutically acceptable carrier for such use.

In a specific embodiment, the disease is atopic dermatitis or allergic dermatitis of dogs.

### DESCRIPTION OF THE FIGURES

FIG. 1 displays the results of Compound 1 when tested in the IL-31 induced itching model.

FIG. 1A shows the comparison of Compound 1 to the placebo and Apoquel.

FIG. 1B shows the effect of three different doses of Compound 1.

### DETAILED DESCRIPTION OF THE INVENTION

The present invention has the object of providing effective and safe compounds for of an JAK-mediated disease that is effective and can be safely administered to animals below the age of 12 months. The current invention addresses this need.

It is to be understood that the invention is not limited to exemplified methods or compositions disclosed herein. It is also to be understood that the terminology used herein is for the purpose of describing particular embodiments of the invention only and is not intended to be limiting.

Before describing the present invention in detail, some terms used in the context of the present invention will be defined. In addition to these terms, others are defined elsewhere in the specification, as necessary.

Unless otherwise expressly defined herein, terms of art used in this specification will have their art-recognized meanings.

As used in the specification and claims, the singular form “a”, “an” and “the” include plural references unless the context clearly dictates otherwise.

As used herein, the term “comprising” is intended to mean that the compositions and methods include the recited elements, but not excluding others.

The inventors found that a compound of Formula (I) can be surprisingly administered to young animals, that are younger than 12 months of age to treat a JAK-1 mediated disease of companion animals such as dogs, cats and horses, especially dogs.

The compound of Formula (I) is described to be a JAK-1 inhibitor. The only commercialized veterinary product with such mode of action is the product Apoquel, a veterinary drug whose active ingredient is oclacitinib which is authorized for the control of pruritus associated with atopic dermatitis and control of atopic dermatitis in dogs at least 12 months in age (See FOI Summary for NADA 141-345, May 14, 2013). See also U.S. Pat. Nos. 6,890,929; 7,687,507; 8,133,899 and 8,987,283.

For Apoquel it was found in a margin of safety study, that the most severe adverse clinical effects were observed in dogs aged less than one year. Some of the effects observed in young dogs (relative to adult animals) may be due to differences in immune system competence. As a result of the effects seen in young dogs, the decision was made to limit its use to dogs of greater than 1 year of age.

Therefore, the only available JAK-1 inhibitor Apoquel is contraindicated for use below 12 months of age.

Age is generally known to be an important factor when considering phenotypic changes in health and disease of animals. A patient's age can affect the course and progression of a disease or can be important in determining the correct course of treatment.

Consequently, based on the available information it would not be expected, that JAK inhibitors could be used successfully in companion animals younger than 12 months.

However, the signs of atopic dermatitis develop already in such young adult animals below the age of 12 months. The age of onset of canine atopic dermatitis is typically between 6 months and 3 years.

Dogs of this age often show symptoms of an atopic syndrome, especially atopic dermatitis or pruritus in connection with allergic dermatitis.

Therefore, it is desirable that animals younger than 12 months, especially with dermatological symptoms (such as pruritus) can be (already) treated at the onset of such symptoms at a young age.

This is very important for the control of progression of such diseases. Early treatment prevents on one hand the worsening of symptoms at this age and on the other hand the appearance of secondary infections by pathogens appearing when the skin barrier of the animal is broken after scratching.

Animals with atopic dermatitis are prone to secondary skin infections, ear infections and Malassezia (yeast) infections and frequently have sensitive skin. Any skin infection, irritating substance, or fleas will aggravate the allergic condition and may cause flare-ups in controlled cases.

Dogs with allergic dermatitis frequently have concurrent infections of the skin and ears. Atopic dogs have a higher abundance of Staphylococcus spp. on their skin, notably Staphylococcus pseudintermedius, which is commonly implicated in pyoderma. Studies infer that staphylococci have increased adherence to inflamed and atopic skin, which could explain the increased abundance of this organism. It has been suggested that these bacteria are also involved in hypersensitivity responses, most commonly due to staphylococcal components or toxins acting as superantigens. Treatment often requires topical and/or systemic antibacterial therapy.

Consequently, pyoderma is often caused by pruritus that has not been treated early enough.

Therefore, a treatment of young animals, especially dogs below 12 months of age would offer a significant benefit for the treatment of atopic dermatitis and allergic dermatitis.

It has been surprisingly found that the compound of Formula (I) can be administered to young animals, especially dogs between approximately 6 and 12 months of age as shown in Example 1 that show that a margin of safety has been established for a therapeutically effective dose of a compound of Formula (I).

The age is the age of the animal at initiation of the treatment and the treatment is continued beyond this age.

It has been found that the compound of Formula (I) effectively controls pruritus in companion animals.

Consequently, in one embodiment the compound of Formula (I) is used in the treatment of pruritus associated with allergic dermatitis in dogs or the treatment of clinical manifestations of atopic dermatitis in companion animals, especially in dogs that are younger than 12 months of age.

In one embodiment the age of the treated animal is between approximately 8 and 12 months of age

The compound of Formula (I) can be used for the control of pruritus associated with allergic dermatitis and control of atopic dermatitis in companion animals, especially in dogs younger than 12 months of age. In one embodiment the age of the treated animal is between approximately 8 and 12 months of age.

One of the objects of the present invention is therefore the use compound of Formula (I), or a pharmaceutical composition or a veterinary product comprising such compound for the manufacturing of a medicament for the treatment of an JAK-1 mediated atopic disease in such young companion animals.

It is especially useful for JAK-1 mediated diseases with pruritic conditions such as atopic dermatitis, allergic dermatitis, eczema, or summer eczema (especially of horses).

In a specific embodiment, the disease is allergic dermatitis or atopic dermatitis. Preferably the disease is atopic dermatitis of dogs or cats, especially dogs.

In a specific embodiment the compound of Formula (I) is used for treatment of clinical manifestations of atopic dermatitis in dogs.

In another embodiment the compound of Formula (I) is used for treatment of pruritus associated with allergic dermatitis in dogs.

The compound for use in the present invention is a compound of Formula (I) or pharmaceutically acceptable salt or stereoisomer thereof:

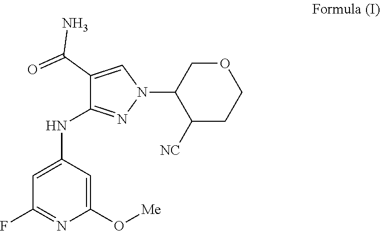 [SMILES: COc1cc(Nc2nn(C3COCCC3C#N)cc2C(N)=O)cc(F)n1]

Compounds of Formula (I) contain one or more asymmetric centers and can thus occur as racemates and racemic mixtures, single enantiomers, diastereomeric mixtures and individual diastereomers. The present invention is meant to comprehend all such isomeric forms of the compounds of Formula I, either as single species or mixtures thereof.

In a preferred embodiment, the compound of Formula I is

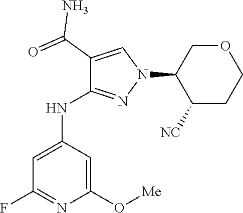 [SMILES: COc1cc(Nc2nn([C@H]3COCC[C@@H]3C#N)cc2C(N)=O)cc(F)n1]

In one embodiment, the compounds of the instant invention are selective JAK-1 inhibitors relative to JAK-2 and JAK-3. In an embodiment, the compounds of the instant invention are selective JAK-1 inhibitors relative to JAK-2 or JAK-3. The determination of relative selectivity for a given compound of JAK1 inhibition is defined as the relative ratio of the (JAK2 IC₅₀ value/JAK1 IC₅₀ value) is at least 2. Also, the relative ratio of the (JAK3 IC₅₀ value/JAK1 IC₅₀ value) is at least 500.

In yet another embodiment, for a given compound, the relative ratios of the (JAK2 IC₅₀ value/JAK1 IC₅₀ value) is at least 5 or is at least 10. In another embodiment, the relative ratio of the (JAK3 IC₅₀ value/JAK1 IC₅₀ value) is at least 500 or is at least 750 or is at least 1000.

The term “pharmaceutically acceptable salts” refers to salts prepared from pharmaceutically acceptable non-toxic bases including inorganic bases and organic bases. When the compound of the present invention is basic, salts may be prepared from pharmaceutically acceptable non-toxic acids, including inorganic and organic acids.

It will be understood that, unless otherwise specified, references to the compound of Formula (I) are meant to also include the stereoisomer thereof or pharmaceutically acceptable salts thereof.

The compound of Formula (I) can be used to treat JAK-1 mediated diseases of companion animals.

In one embodiment the JAK-1 mediated disease is one that can be ameliorated by the selective inhibition of a Janus kinase JAK-1 relative to JAK 2 and JAK 3.

Important known veterinary JAK-1 mediated diseases include conditions or diseases are e.g., allergic diseases or conditions of the skin, gastrointestinal and respiratory tract. Examples are allergic reactions, allergic dermatitis, atopic dermatitis, eczema, summer eczema, urticaria, heaves, inflammatory airway disease, recurrent airway obstruction, airway hyper-responsiveness, chronic obstructive pulmonary disease, inflammatory processes resulting from autoimmunity and inflammatory diseases of non-human animals, especially of companion animals.

In a specific embodiment, the JAK-1 mediated disease is allergic dermatitis or atopic dermatitis. Preferably the disease is atopic dermatitis of companion animals, especially dogs or cats, preferably dogs.

These diseases are related to an allergic reaction. The term “allergic reaction” is defined herein as a disorder or disease caused by an interaction between the immune system and a substance foreign to the body. This foreign substance is termed “an allergen”. Common allergens include aeroallergens, such as pollens, dust, molds, dust mite proteins, injected saliva from insect bites, etc. Atopic dermatitis is the second most common allergic skin condition in canines, surpassed only by flea allergies.

A number of JAK-1 mediated diseases include a pruritic condition. Examples of JAK-1 mediated diseases that include pruritic conditions include but are not limited to the following: atopic dermatitis, allergic dermatitis, eczema, and summer eczema.

Companion animals with atopic or allergic dermatitis often suffer from pruritus, hair loss, excoriation of the skin from deep scratching, frequent licking of their paws and excessive tear production.

Pruritus is a sensation and is synonymous with itch. Animals generally display the sensation of itch clinically by scratching. However, it may also be manifested by licking, sucking, biting, chewing, rubbing, or rolling. When the pruritus remains uncontrolled, especially over a longer period often secondary skin conditions complicate the treatment.

Skin conditions resulting from pruritus in animals can be e.g., alopecia, scaling, hyperpigmentation, erythema, crusting, papules, and pustules. In younger dogs (or old dogs with a longstanding history dating from an onset of the disease as a young animal), it may present as chronic or chronic relapsing pyoderma.

It has been found that the compound of Formula (I) effectively controls pruritus as shown in Example 2.

Consequently, in one embodiment the compound of Formula (I) can be used in the treatment of pruritus associated with allergic dermatitis in dogs. The compound of Formula (I) can be used in the treatment of clinical manifestations of atopic dermatitis in companion animals, especially in dogs.

The compound of Formula (I) can be used for the treatment or control of pruritus associated with allergic dermatitis and control of atopic dermatitis in companion animals, especially in dogs younger than 12 months of age.

The companion animal is a canine (e.g., domestic dog), feline (e.g., house cat), equine (e.g., horse). Preferred is the treatment of a dog or cat, especially a dog.

Also as used herein, the terms “treat,” “treating” or “treatment” refer to any type of action that imparts a modulating effect, which, for example, can be a beneficial and/or therapeutic effect, to a animal afflicted with a condition, disorder, disease or illness, including, for example, improvement in the condition of the subject (e.g., in one or more symptoms), delay in the progression of the disorder, disease or illness, and/or change in clinical parameters of the condition, disorder, disease or illness, etc., as would be well known in the art.

Additionally, as used herein, the terms “prevent,” “preventing” or “prevention” refer to any type of action that results in the absence, avoidance and/or delay of the onset and/or progression of a disease, disorder and/or a clinical symptom(s) in an animal and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the animal and/or the severity of onset is less than what would occur in the absence of the present invention.

The term treatment” in the current invention includes aspects of prevention as described above.

A “therapeutically effective dose” or “therapeutically effective amount” refers to an amount of a compound or composition of this invention that is sufficient to produce a desired effect, which can be a therapeutic and/or beneficial effect. It is an amount that is non-toxic to the animal, and sufficient to achieve the desired effect, by reducing the signs and symptoms associated with a disease or disorder.

For allergic diseases such effect is e.g., a reduction or elimination in the severity and/or frequency of symptoms and/or improvement or remediation of damage (e.g., a reduction, delay, and/or prevention of flares) and/or reducing, inhibiting, relieving or preventing lesions and/or itch in an animal with atopic or allergic dermatitis.

Non-toxic in this document means that at the specific dosage there are no severe side reactions in the treated animal. This means specifically, if side effects are observed they are acceptable, e.g., clinical or microscopic findings are temporary and resolve without requiring (long) treatment or other intervention or show only a small magnitude of change (such as for hematology parameters).

Another important aspect for such a chronic disease is that the treatment with the therapeutically effective and non-toxic dosage of the compound of Formula (I) that controls pruritus results in a significant improvement of the quality of life of the animal (and the pet owner). This is also important from an animal welfare standpoint because such a chronic disease results in a continuous discomfort of the animal. Especially for young dogs between the age of approximately 6 months and 12 months continuous itching disturbs mental and physical development, can result in undesired behavior of the animal and can negatively impact socialization of the young (dog) animal.

It has been surprisingly observed that a therapeutically effective dosage was well tolerated and was non-toxic in young dogs that are below the age of 12 months. This finding was unexpected in view of the reported side effects that were observed with the commercially available JAK-inhibitor Apoquel in their study with young dogs that resulted in serious side effects and a contraindication for use in dogs younger than 12 months.

The therapeutically effective daily dosage of the compound of Formula (I) is from about 0.1 mg/kg to about 6.0 mg/kg. In one embodiment at the therapeutically effective dose is from about 0.25 mg/kg to about 3.0 mg/kg per day, preferably 0.5 to about 1.8 mg/kg body weight per day or about 0.6 mg/kg to about 1.2 mg/kg body weight or alternatively 1 mg/kg bodyweight.

Preferably a therapeutically effective dose of the compound of Formula (I) is administered orally to the animal. This has the advantage that the administration does not require the intervention of a veterinarian and can be continued over a long period of time.

Atopic dermatitis or allergic dermatitis often require a lifelong treatment of this condition. In many embodiments, treatment will continue on a consecutive daily basis (as a maintenance therapy) for many weeks, if not for the rest of the animal's life.

Typically, treatment is continued with daily administration repeated for at least 14 consecutive days, if not longer, i.e., for at least a month, preferably at least four months. In many embodiments, daily treatment is continued for at least a year, if not longer, i.e., 5 years to the rest of the animal's life.

In one embodiment the administration is repeated daily for at least four months.

In another embodiment the administration is repeated daily for at least a year.

The desired therapeutically effective dose may conveniently be presented in a single daily dose or as divided doses administered at appropriate intervals, for example, as two, three, four or more sub-doses per day

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

The daily dose may also be divided into multiple doses for administration, e.g., two times per day e.g., the daily dose is split into % of the daily dose that is administered twice per day approximately 12 hours apart.

In one embodiment a dose of the compound of Formula (I) is administered initially twice daily for two to six weeks and the same dose as maintenance therapy once daily thereafter.

In one preferred embodiment the compound of Formula (I) is administered orally, twice daily for up to 14 days, preferably 2 weeks, and then administered once daily for maintenance therapy, that may last for 4 months, a year or longer.

In another preferred embodiment a therapeutically effective dose of the compound of Formula (I) is administered orally, twice daily for 6 weeks and then the same dose is administered once daily for maintenance therapy.

In some embodiments, the compound of Formula (I) is administered in combination with one or more additional active ingredients. Such additional active ingredient can be e.g., an immunomodulator, anti-inflammatory agent or an antibiotic.

Compounds of Formula (I) may be administered in a pharmaceutically acceptable form either alone or in combination with one or more additional active ingredients, which modulate a mammalian immune system or with anti-inflammatory agents e.g., NSAIDs or anti-inflammatory steroids (e.g., prednisolone or dexamethasone). These agents may be administered as part of the same or separate dosage forms, via the same or different routes of administration, and on the same or different administration schedules according to standard pharmaceutical practice known to one skilled in the art.

Preferably the compound of Formula (I) is administered in a pharmaceutical composition. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients.

Accordingly, the pharmaceutical compositions of the present invention encompass any composition made by admixing a compound of Formula (I), and at least one pharmaceutically acceptable excipient.

In various embodiments, the compound of Formula (I) is formulated in unit dose form as a tablet or capsule. In some embodiments, the tablet or capsule is chewable. Additionally, while the texture and formulation of the unit dose form for cats is generally the same or substantially similar to that for the dog, the cat unit dose form may be made available in smaller sizes and/or concentrations more appropriate for smaller animals.

In one embodiment the pharmaceutical composition for use in the current invention is a unit dose for oral administration, preferably a tablet comprising a flavoring that is palatable to an intended companion animal.

In various embodiments, the compound of Formula (I) is formulated in a composition that comprises a flavoring palatable to an intended companion animal. For example, in some instances a formulation is provided comprising a flavoring palatable to a canine. In other instances, a formulation is provided comprising a flavoring palatable to a feline. Suitable flavorings include beef, chicken, pork, fish, and turkey flavorings, or any other flavoring used for canine and feline foods. In one embodiment, the flavoring is a preparation comprising one or more of beef liver, chicken liver, pork liver, and turkey liver.

In one embodiment, the present invention provides an orally administered, chewable, flavored pharmaceutical composition of the compound of Formula (I) for the treatment of atopic or allergic dermatitis in companion animals, for example dogs or cats, preferably dogs. Preferably such companion animals are younger than 12 months of age at initiation of the treatment.

The present invention also provides pharmaceutical formulations in the form of pastes and gels that contain a therapeutically effective dose of the compound of Formula (i). In one embodiment, especially suitable for administration to a horse, the appropriate dose is administered in the form of a paste that is applied to the horse's gums and/or teeth. In another embodiment, especially suitable for administration to a cat, the appropriate dose is administered in the form of a paste that is applied to the cat's paws and/or fur.

Such pharmaceutical compositions can be in general prepared using standard formulation processes and equipment and can comprise in addition to a compound of Formula (I) an additional active ingredient, e.g., as described earlier.

### EXAMPLES

In the following Examples Compound 1 is 1-((3R,4S) or (3S,4R)-4-cyanotetrahydro-2H-pyran-3-yl)-3-((2-fluoro-6-methoxypyridin-4-yl)amino)-1H-pyrazole-4-carboxamide).

### Example 1

A pilot Target Animal Safety study has been performed.

The objective of this study was to provide margin of safety information on Compound 1 at 1, 3 and 5 times the maximum recommended commercial dose (0.6 mg/kg body weight twice daily for two weeks and once daily thereafter (BID), or 1.2 mg/kg body weight daily (SID)) in 8-month-old Beagle dogs (at initiation of dosing), when given orally for 4 months (112 consecutive days). The clinical loading phase of 2 weeks for the BID treatment schedule was prolonged to 6 weeks in this study in accordance with VICH GL43 (3 times the proposed duration).

The test item was the final formulation blend of the investigational veterinary product, filled into gelatine capsules for dose administration. The control article was blank (empty) gelatine capsules.

In addition to that, a 4-month pilot target animal safety study has been conducted in 6-months old dogs utilizing either doses of 0.6, 1.8 or 3.0 mg/kg twice daily over the first 6 weeks (loading phase) and reducing to once daily thereafter (BID), or a dose of 6.0 mg/kg administered once daily over the whole study period (SID) compared to a control group, using 3 males and 3 females per group (Robertson, 2021).

All doses were well tolerated by male and female dogs with no changes in body weight, food consumption, ophthalmic examinations, electrocardiology or blood pressure changes.

It can be concluded that the daily oral administration of Compound 1 at up to 5 times the recommended maximum commercial dose of 1.2 mg/kg/body weight (BID or SID dosing) was well tolerated in male and female 6-month-old beagle dogs. Minor clinical findings (e.g., interdigital cysts, conjunctivitis, gingivitis, diarrhoea) in overdosed animals were observed. The cysts had no impact on the overall function of the musculoskeletal system (lameness). Moreover, the other findings mentioned above resolved under veterinary treatment. Minor infections like conjunctivitis, gingivitis, diarrhoea as well as interdigital cysts can generally be a recurrent problem in large dog colonies and are often associated with environmental germs (Ledbetter et al, 2009; Kovacs et al, 2005; Saleh et al, 2016).

In summary, even repeated overdoses with compound 1 only led to increased susceptibility to minor infections (e.g., conjunctivitis, gingivitis, diarrhoea, interdigital cysts) associated with environmental germs. At the recommended maximum commercial dose, no increased susceptibility to minor infections was observed.

Therefore, it is concluded that the compound 1 is considered to be safe for use in young dogs.

### Example 2

A canine interleukin-31 (cIL-31) induced pruritus model in the Beagle dog was used to inform on the dose of Compound 1 required to inhibit JAK1 signaling in vivo. The cIL-31 induced pruritus model is a relevant model of acute pruritus associated with atopic and allergic dermatitis in dogs. The magnitude of the suppression of cIL-31 induced pruritus by JAK inhibitors is predictive of their pruritus suppression in patients with allergic or atopic dermatitis As a measure of JAK-1 inhibition in vivo, and by extension the anticipated clinical efficacy of Compound 1, we evaluated Compound 1 in a relevant pharmacodynamic model and included a clinical reference. Canine interlukin-31 (cIL-31) has been demonstrated to be involved in the pruritus associated with atopic and allergic dermatitis in dogs [Gonzales et al., Vet Dermatol 2013; 24: 48], and IL-31 can activate JAK-1 and JAK-2 signaling molecules after binding to its receptor complex [Zhang et al., Cytokine & Growth Factor Reviews 19 (2008) 347-356]. cIL-31 administration to Beagle dogs produces a robust pruritic response that can be inhibited by prior treatment with the JAK inhibitor oclacitinib [Gonzales et al., Vet Dermatol 2016; 27: 34-e10].

Using a randomized, non-blinded, cross-over study design, Compound 1 (1 mg/kg body weight), Apoquel®, or placebo was dosed orally to laboratory Beagle dogs 2 h prior to a cIL-31 challenge (approximate Tₘₐₓ of Compound 1 and Apoquel®). Dogs were observed for 2 h after cIL-31 challenge, and the time animals were engaged in pruritic behaviors was recorded.

In this study, Compound 1 significantly suppressed the cIL-31 induced pruritus; the magnitude was similar to Apoquel. In a second study using a randomized, non-blinded crossover design, several doses of Compound 1 were evaluated (0.5, 0.1 and 0.05 mg/kg body weight); Apoquel® and placebo treatments were also included.

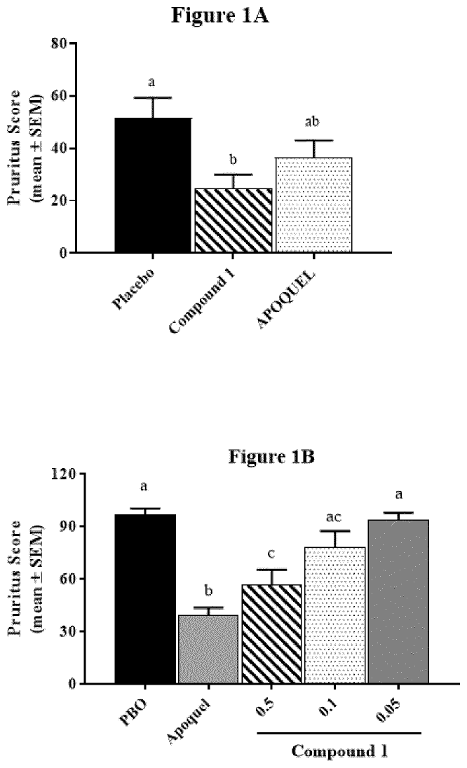

Compound 1 significantly suppressed pruritus at the 0.5 mg/kg body weight dose, but not at the 0.1 and 0.05 mg/kg body weight doses. The magnitude of effect was similar between 0.5 mg/kg Compound 1 and Apoquel®. See FIGS. 1A and 1B.

1. A method of treating a JAK-1 mediated disease of a companion animal comprising administering to the companion animal an effective dose of a compound of Formula (I) or a pharmaceutically acceptable salt or stereoisomer thereof 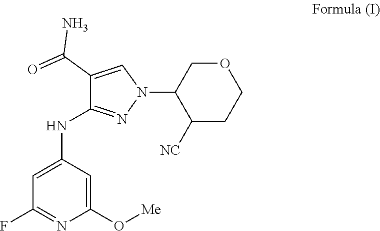 [SMILES: COc1cc(Nc2nn(C3COCCC3C#N)cc2C(N)=O)cc(F)n1]
wherein the companion animal is selected from dog, cat, and horse and is younger than 12 months of age.

2. The method according to claim 1, characterized in that the compound of Formula (I) is

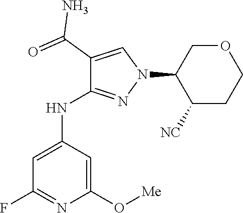 [SMILES: COc1cc(Nc2nn([C@H]3COCC[C@@H]3C#N)cc2C(N)=O)cc(F)n1]

3. The method according to claim 1, characterized in that the animal is between approximately 6 and 12 months of age.

4. The method according to claim 1, characterized in that the animal is between approximately 8 and 12 months of age.

5. The method according to claim 1, characterized in that the animal is a dog.

6. The method according to claim 1, characterized in that the JAK-1 mediated disease is allergic dermatitis or atopic dermatitis.

7. The method according to claim 6, wherein the JAK-1 mediated disease is atopic dermatitis.

8. The method according to claim 6, wherein the JAK-1 mediated disease is allergic dermatitis.

9. The method according to claim 1, characterized in that the therapeutically effective dose of the compound is from about 0.1 mg/kg to about 6.0 mg/kg per day.

10. The method according to claim 9, characterized in that the therapeutically effective dose is from about 0.25 mg/kg to about 3.0 mg/kg per day.

11. The method according to claim 9, characterized in that the therapeutically effective dose is from about 0.6 mg/kg to about 1.2 mg/kg body weight per day.

12. The method according to claim 1, characterized in that the therapeutically effective dose is administered orally.

13. The method according to claim 1, characterized in that the administration is repeated daily for at least four months.

14. The method according to claim 13, characterized in that the administration is repeated daily for at least a year.

15. The method according to claim 1, characterized in that a dose of the compound is administered initially twice daily for two to six weeks and once daily thereafter.

16. The method according to claim 1, characterized in that the compound of Formula (I) is administered in combination with one or more additional active ingredients.

17. The method according to claim 16, characterized in that such additional active ingredient is an immunomodulator, anti-inflammatory agent or an antibiotic agent or antifungal agent.

18-19. (canceled)

20. The method according to claim 1 wherein the compound of Formula (I) is administered in combination with a flavoring that is palatable to an intended companion animal.

21. The method of claim 10 wherein the therapeutically effective dose is from about 0.6 to about 1.8 mg/kg body weight per day.
